(19) United States
(12) Patent Application Publication
(10) Pub. No.: US 2009/0082745 A1
(43) Pub. Date: Mar. 26, 2009

(54) SANITARY ARTICLE FOR ABSORBING EXCRETIONS OF HUMAN BODY

(76) Inventors: Nai-Ko YING, Taipei City (TW); Jeng-Homg HOU, Taipei City (TW)

Correspondence Address: HDLS Patent & Trademark Services, P.O. BOX 220746, CHANTILLY, VA, 20153-0746, US

(21) Appl. No.: 12/056,404

(22) Filed: Mar. 27, 2008

(30) Foreign Application Priority Data
Apr. 4, 2007 (TW) 096112039

(51) Int. Cl. A61F 13/15; A61B 5/00

(52) U.S. Cl. 604/367; 604/385.101; 604/385.23; 600/301; 600/573; 604/378

(57) ABSTRACT

A sanitary article that is used to cover the crotch portion of a human body so as to prevent excretions of the human body from leaking to the outside includes a liquid-permeable surface thin layer, a liquid-impermeable back thin layer and a liquid-absorptive core layer sandwiched between the above two layers. The liquid-absorptive core layer is provided with a draining means for allowing some of the excretions of the human body to pass through. A detecting body is provided between the liquid-impermeable back thin layer and the liquid-absorptive core layer, thereby receiving the excretions guided by the draining means. A window portion is provided on the liquid-impermeable back thin layer at a position corresponding to the detecting body. Via this arrangement, the use can observe the reaction represented by the internal detecting body directly via the window portion.

### BACKGROUND OF THE INVENTION

1. Field of the Invention

The present invention relates to a personal sanitary article, and in particular to a sanitary article that is capable of absorbing excretions of a human body and has a function of physiological detection.

2. Description of Prior Art

Common sanitary articles, such as diaper pants for infants or adults or sanitary napkins for women, are used to cover the crotch portion of a human body so as to prevent the leakage of body fluid, such as urine, feces or menstrual blood, thereby removing the excretions of infants or elders and keeping women clean during the menstrual period. Alternatively, the above sanitary articles can be used to absorb the body fluid and excretions, thereby achieving a brief effect of isolation. In this way, it can reduce the possible direct contact with the skin that may make a user inconvenient or affect the health thereof.

Furthermore, according to the viewpoint of medical test, the excretions such as urine or feces excreted by the human body and the body fluid such as menstrual blood of a woman can be used to detect whether the condition of the human body is normal or whether any pathological change has generated, such as the detections of PH value, leukocyte, nitrites, proteins, glucose, ketones, urobilinogen, bilirubin, hemoglobin, stool urobilinogen and microorganisms. In this way, the pathological problem of the human body can be found at an earlier stage and thus a proper treatment can be performed, thereby preventing diseases.

Therefore, all of the conventional sanitary articles have a leakage-proof effect, and in addition, some of them have a function of physiological detection. The user can understand his/her physical condition based on the excretions thereof, thereby finding out some pathological problems at an earlier stage and performing suitable diagnosis or treatments. However, among the current sanitary articles having a physiological detection, a test paper or test piece suitable for detecting the excretions is provided on the inside of the sanitary article, i.e. the excretions-receiving portion of the sanitary article. As a result, when the user intends to observe the test paper or test piece, the user cannot choose but remove the excretions first. Thus, it is very inconvenient and not sanitary for the user.

Therefore, in view of this, the inventor proposes the present invention to overcome the above problems based on his expert experience and deliberate researches.

### SUMMARY OF THE INVENTION

The present invention is to provide a sanitary article for absorbing excretions of a human body. Via this arrangement, from the outside of the sanitary article, the user can directly observe a test paper or test piece used for detecting the excretions, thereby conforming to the requirements for hygiene and convenience.

In order to achieve the above objects, the present invention provides a sanitary article for absorbing excretions of a human body, which includes a liquid-permeable surface thin layer, a liquid-impermeable back thin layer and a liquid-absorptive core layer sandwiched between the above two layers. The liquid-absorptive core layer is provided with a draining means for allowing some of the excretions of the human body to pass through. A detecting body is provided between the liquid-impermeable back thin layer and the liquid-absorptive core layer, thereby receiving the excretions guided by the draining means. A window portion is provided on the liquid-impermeable back thin layer at a position corresponding to the detecting body. Via this arrangement, the use can observe the reaction represented by the internal detecting body directly via the window portion, thereby understanding the physical condition thereof.

### BRIEF DESCRIPTION OF THE DRAWINGS

FIG. 1 is a schematic view showing that the present invention is applied to a diaper pants;

FIG. 2 is a cross-sectional view taken along the line 2-2 in FIG. 1;

FIG. 3 is a schematic view showing that the excretions of a human body is guided to the detecting piece according to the present invention; and

FIG. 4 is a schematic view showing that the present invention is applied to a sanitary napkin.

### DETAILED DESCRIPTION OF THE INVENTION

In order to make the Examiner to better understand the characteristics and technical contents of the present invention, a detailed description will be made with reference to the accompanying drawings. However, it should be understood that the drawings are illustrative only but not to limit the present invention thereto.

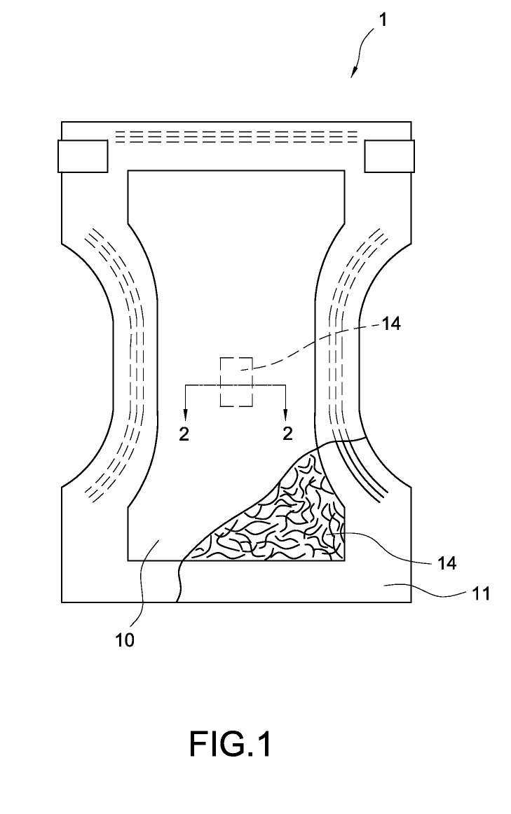

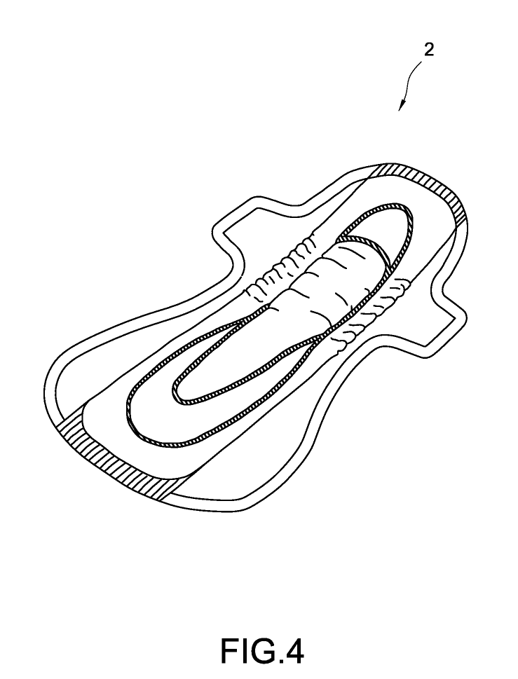

FIG. 1 is a schematic view showing that the present invention is applied to a diaper pants, and FIG. 4 is a schematic view showing that the present invention is applied to a sanitary napkin. The present invention provides a sanitary article for absorbing excretions of a human body, which can be applied to a diaper pants (FIG. 1) for infants or adults or a sanitary napkin (FIG. 4) for women. The sanitary article is used to cover the crotch portion of the human body, thereby absorbing and preventing the body fluid or excretions of the human body from leaking to the outside. Further, the sanitary article of the present invention also has a function of detecting the physical or physiological condition.

As shown in FIG. 1, it is a schematic view showing that the present invention is applied to a diaper pants. In the present embodiment, the sanitary article is a diaper paints 1. The diaper paints 1 comprises a liquid-permeable surface thin layer 10, a liquid-impermeable back thin layer 11 and a liquid-absorptive core layer 12 sandwiched between the above two layers 10, 11. The liquid-absorptive core layer 12 further comprises a high-speed absorptive layer 120 and a low-speed absorptive layer 121 that are overlapped with each other. The profile of the diaper paints in which the front and rear portions thereof are wide but the middle portion thereof is narrow is suitable to be worn on the crotch portion of the human body. Since the structure of the above-mentioned diaper paints 1 is conventional and this part is not the subject matter claimed by the present invention. Accordingly, the detailed description relating to the diaper paints 1 is omitted for clarity.

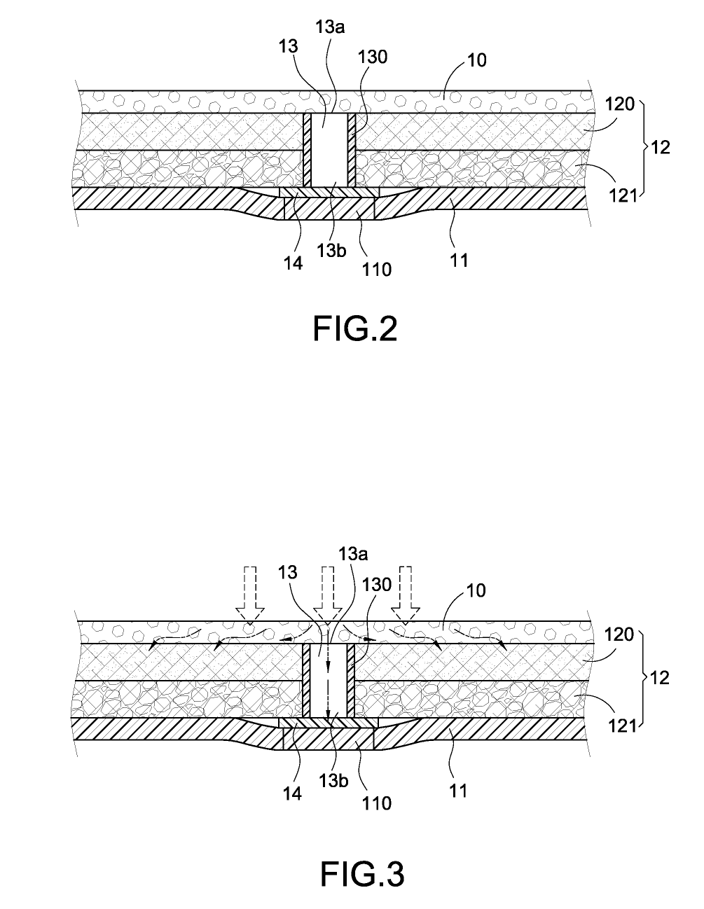

With reference to FIG. 2, it is a cross-sectional view taken along the line 2-2 in FIG. 1. The present invention lies in that the liquid-absorptive core layer 12 of the diaper paints 1 is provided with a draining means 13 for allowing some of the excretions of the human body to pass through the liquid-absorptive core layer 12. The draining means 13 has an inlet 13a adjacent to the liquid-permeable surface thin layer 10 and an outlet 13b adjacent to the liquid-impermeable back thin layer 11. Furthermore, a detecting body 14 is provided between the liquid-impermeable back thin layer 11 and the liquid absorptive core layer 12 to face the outlet 13b of the draining means 13. The detecting body 14 is used to receive and detect the excretions (such urine or feces) of the user. The detecting body can be a common test paper or a test piece made of other materials. Further, a window portion 110 is provided on the liquid-impermeable back thin layer 11 to face the detecting body 14. The window portion 110 can be formed by means of hollowing out the above-mentioned position and adhering a transparent film, so that the user can observe the change of the detecting body 14 from the outside of the liquid-impermeable back thin layer 11 via the window portion 110. In general, the user can recognize the color represented by the detecting body 14 after the detecting body has contacted the excretion of the user.

As shown in FIG. 3, when the user excretes the excretions such as urines or feces in the diaper paints 1, although most of the liquid contents of the excretions are absorbed by the liquid-absorptive core layer 12 via the liquid-permeable surface thin layer 10, a little portion thereof can be still guided to the detecting body 14 via the draining means 13 so as to contact the detecting body 14 to generate a reaction. The user can observe the change of the detecting body 14 from the outside of the liquid-impermeable back thin layer 11 via the window portion 110. Furthermore, according to the present invention, the inner peripheral wall of the draining means 13 is provided with an annular blocking layer 130, thereby avoiding the liquid excretions from being completely absorbed by the liquid-absorptive core layer 12 via the inner peripheral wall of the draining means 13 when the amount of the liquid excretions is too small.

With reference to FIG. 4, it is a schematic view showing that the present invention is applied to a sanitary napkin. The present invention can be a sanitary napkin 2 in which the detecting body 14 is used to contact the menstrual blood and associated excretions to generate a reaction during the menstrual period of a woman. The user can observe the detecting body 14 via the window portion 110, thereby recognizing whether the detection result is normal or not and understanding the physical condition thereof.

Therefore, with the above construction, the sanitary article for absorbing excretions of a human body in accordance with the present invention can be obtained.

According to the above, the present invention indeed achieves the desired effects and solves the drawbacks of prior art. Further, the present invention really has novelty and inventive steps and thus conforms to the requirements for an invention patent.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

1. A sanitary article for absorbing excretions of a human body, comprising:
a liquid-permeable surface thin layer;
a liquid-impermeable back thin layer provided with a window portion,
a liquid-absorptive core layer sandwiched between the liquid-permeable surface thin layer and the liquid-impermeable back thin layer, a draining means being provided on the liquid-absorptive core layer for allowing some of the excretions of the human body to pass through; and
a detecting body provided between the liquid-impermeable back thin layer and the liquid-absorptive core layer for receiving the excretions guided by the draining means;
wherein the window portion is provided on the liquid-impermeable back thin layer at a position corresponding to the detecting body.

2. The sanitary article for absorbing excretions of a human body according to claim 1, wherein the liquid-absorptive core layer further comprises a high-speed absorptive layer and a low-speed absorptive layer that are overlapped with each other.

3. The sanitary article for absorbing excretions of a human body according to claim 1, wherein the draining means has an inlet adjacent to the liquid-permeable surface thin layer and an outlet adjacent to the liquid-impermeable back thin layer.

4. The sanitary article for absorbing excretions of a human body according to claim 3, wherein the inner peripheral wall of the draining means is provided with an annular blocking layer.

5. The sanitary article for absorbing excretions of a human body according to claim 1, wherein the detecting body is a test paper.

6. The sanitary article for absorbing excretions of a human body according to claim 1, wherein the detecting body is a test piece.

7. The sanitary article for absorbing excretions of a human body according to claim 1, wherein the sanitary article is a diaper paints.

8. The sanitary article for absorbing excretions of a human body according to claim 1, wherein the sanitary article is a sanitary napkin.
